(19) United States
(12) Patent Application Publication
(10) Pub. No.: US 2010/0291598 A1
(43) Pub. Date: Nov. 18, 2010

(54) PRION ELISA

(76) Inventor: David Peretz, El Cerrito, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/450,634

(22) PCT Filed: Apr. 4, 2008
(86) PCT No.: PCT/US2008/004457

§ 371 Date: Apr. 30, 2010

Related U.S. Application Data
(60) Provisional application No. 60/921,920, filed on Apr. 4, 2007.

(51) Int. Cl. G01N 33/53

(52) U.S. Cl. 435/7.92

(57) ABSTRACT

Assays for detecting PrP^{Sc} in a sample are described. In particular, pathogenic prion ELISAs are described. The assays utilize pathogenic prion-specific reagents to capture the PrP^{Sc} and digestion with a site-specific protease, for example trypsin or SV-8 protease, to reduce the amount of interference from non-pathogenic prion proteins that are occasionally present in the samples.

### TECHNICAL FIELD

This disclosure relates to assays for detecting pathogenic prion proteins in a sample.

### BACKGROUND

In humans, prion diseases, also known as, “transmissible spongiform encephalopathies” (TSEs), include, Creutzfeldt-Jakob disease (CJD), Gerstmann-Straussler-Scheinker syndrome (GSS), Fatal Familial Insomnia, and Kuru (see, e.g., Isselbacher et al., eds. (1994). Harrison's Principles of Internal Medicine. New York: McGraw-Hill, Inc.; Medori et al. (1992) N. Engl. J. Med. 326: 444-9). In animals, TSEs include sheep scrapie, bovine spongiform encephalopathy (BSE), transmissible mink encephalopathy, and chronic wasting disease of captive mule deer and elk (Gajdusek, (1990). Subacute Spongiform Encephalopathies: Transmissible Cerebral Amyloidoses Caused by Unconventional Viruses. In: Virology, Fields, ed., New York: Raven Press, Ltd. (pp. 2289-2324)). Transmissible spongiform encephalopathies are characterized by the same hallmarks: the presence of the abnormal (beta-rich, proteinase K resistant) conformation of the prion protein that transmits disease when experimentally inoculated into laboratory animals including primates, rodents, and transgenic mice.

Recently, the rapid spread of BSE and its correlation with elevated occurrence of TSEs in humans has led to increased interest in the detection of TSEs in non-human mammals. The tragic consequences of accidental transmission of these diseases (see, e.g., Gajdusek, Infectious Amyloids, and Prusiner Prions In Fields Virology. Fields, et al., eds. Philadelphia: Lippincott-Ravin, Pub. (1996); Brown et al. Lancet, 340: 24-27 (1992)), decontamination difficulties (Asher et al. (1986) In: Laboratory Safety: Principles and Practices, Miller ed., (pp. 59-71) Am. Soc. Micro.), and concern about BSE (British Med. J. (1995) 311: 1415-1421) underlie the urgency of having a diagnostic test that would identify humans and animals with TSEs.

Prions differ significantly from bacteria, viruses and viroids. The dominating hypothesis is that, unlike all other infectious pathogens, infection is caused by an abnormal conformation of the prion protein, which acts as a template and converts normal prion conformations into abnormal, aberrant conformations. A prion protein was first characterized in the early 1980s. (See, e.g., Bolton, McKinley et al. (1982) Science 218: 1309-1311; Prusiner, Bolton et al. (1982) Biochemistry 21: 6942-6950; McKinley, Bolton et al. (1983) Cell 35: 57-62). Complete prion protein-encoding genes have since been cloned, sequenced and expressed in transgenic animals. (See, e.g., Basler, Oesch et al. (1986) Cell 46: 417-428.)

The key characteristic of prion diseases is the formation of the abnormally shaped protein (PrP^{Sp}) from the normal form of prion protein (cellular or nonpathogenic or PrP^{C}). (See, e.g., Zhang et al. (1997) Biochem. 36(12): 3543-3553; Cohen & Prusiner (1998) Ann. Rev. Biochem. 67: 793-819; Pan et al. (1993) Proc. Natl. Acad. Sci. USA 90:10962-10966; Safar et al. (1993) J Biol. Chem. 268: 20276-20284.) The substantially β-sheet structure of PrP^{Sp} as compared to the predominantly α-helical folded non-disease forms of PrP^{C} has been revealed by optical spectroscopy and crystallography studies. (See, e.g., Wille et al. (2001) Proc. Nat'l Acad. Sci. USA 99: 3563-3568; Peretz et al. (1997) J. Mol. Biol. 273: 614-622; Cohen & Prusiner, (1999) 5: Structural Studies of Prion Proteins. In Prion Biology And Diseases, S. Prusiner, ed. Cold Spring Harbor, N.Y.: Cold Spring Harbor Laboratory Press. (pp: 191-228.) The structural changes appear to be followed by alterations in biochemical properties: PrP^{C} is soluble in non-denaturing detergents, PrP^{Sp} is insoluble; PrP^{C} is readily digested by proteases, while PrP^{Sp} is partially resistant, resulting in the formation of an amino-terminally truncated fragment known as “PrPres” (Baldwin et al. (1995) J. Biol Chem 270:19197; Tateishi et al. (2002) Nature 376:434), “PrP 27-30” (27-30 kDa) or “PK-resistant” (proteinase K resistant) form. The difference in protease sensitivities has been used to distinguish between the PrP^{SC} and PrP^{C} forms. (See, Prusiner (1998) Proc. Natl. Acad. Sci. 95:13363; Aguzzi (2006) J. Neurochem. 97:1726) Proteinase K completely degrades the PrP^{C} form of the prion protein in 30 minutes at 50 μg/ml, while the PrP^{SC} form maintains a protease resistant core under the same conditions. This protease resistant core of PrP^{SC} includes amino acids from about residue 89 or 90 to about residue 231. The N-terminal region of the PrP^{SC} form, which is more available to proteinase K, is typically removed by proteinase K treatment. Although the PrP^{SC} is fairly resistant to protease digestion, prolonged exposure and/or high concentrations of proteinase K will result in more complete digestion of PrP^{SC}. A protease sensitive form of PrP^{SC} has been reported (Safar et al. (1998) Nature Med. 4:1157).

Despite the difference in some physical properties between the disease form and the normal cellular form of the prion protein, detection of the pathogenic isoforms of prion protein in living subjects, and samples obtained from living subjects, has proven difficult. One reason for this is that, typically, antibodies generated against prion peptides recognize both denatured PrP^{SC} and PrP^{C} but are unable to selectively recognize infectious (undenatured) PrP^{SC}. (See, e.g., Matsunaga et al. (2001) Proteins: Structure, Function and Genetics 44: 110-118). Thus, definitive diagnosis and palliative treatments for these transmissible and amyloid-containing conditions before death of the subject remains a substantially unmet challenge. Histopathological examination of brain biopsies is risky to the subject and lesions and amyloid deposits can be missed depending on where the biopsy sample is taken from. Also, there are still risks involved with biopsies to animals, patients, and health care personnel. Further, the results from brain tests on animals are not usually obtained until the animal has entered the food supply.

Even so, a number of post-mortem tests for TSE are available (See, Soto, C. (2004) Nature Reviews Microbiol. 2:809, Biffiger et al. (2002) J. Virol. Meth. 101:79; Safar et al. (2002) Nature Biotech. 20:1147, Schaller et al. Acta Neuropathol. (1999) 98:437, Lane et al. (2003) Clin. Chem. 49:1774). However, all of these utilize brain tissue samples and are suitable only as post-mortem tests. Most of these require proteinase K treatment of the samples as well, which can be time-consuming, incomplete digestion of PrP^{C} can lead to false positive results, and digestion of PK-sensitive PrP^{Sc} can yield false negative results.

Thus, there remains a need for compositions and methods for detecting the presence of the pathogenic prion proteins in various samples, for example in samples obtained from living subjects, in blood supplies, in farm animals and in other human and animal food supplies. This disclosure is directed to these, as well as other, important ends.

### SUMMARY

The present disclosure relates to improvements in recently described methods for detecting the presence of prion proteins. These detection methods are described herein and in co-owned applications, U.S. application Ser. No. 10/917,646, filed 13 Aug. 2004; U.S. application Ser. No. 11/056,950, filed 11 Feb. 2005; U.S. application Ser. No. 11/518,091, filed 8 Sep. 2006; and International application No. PCT/US2006/001433, filed 13 Jan. 2006, all of which applications are incorporated herein by reference in their entireties. The detection methods may be used, inter alia, in connection with methods for diagnosing a prion-related disease (e.g., in human or non-human animal subjects), for ensuring a substantially PrP^{Sc}-free blood supply, blood products supply, or food supply, for analyzing organ and tissue samples for transplantation, for monitoring the decontamination of surgical tools and equipment, as well as any other situation in which knowledge of the presence or absence of the pathogenic prion is important. The detection methods take advantage of the preferential interaction of prion-specific reagents with the pathogenic prion isoform. In general, the prion-specific reagent, which can be a peptide reagent as described in U.S. application Ser. No. 10/917,646 and U.S. application Ser. No. 11/056,950, a peptoid reagent as described in U.S. application Ser. No. 11/518,091 and WO2007/030804, or other reagents variously described in WO03/085086, WO03/073106, or WO02/097444, is used to bind specifically to the pathogenic form of a prion protein, resulting in a complex which can be separated from the rest of the sample, including from most or all of the non-pathogenic form of the prion protein that may be present in the sample. Once all of the non-pathogenic form of the prion protein has been removed, the remaining pathogenic form of the prion protein can be detected, for example the pathogenic form of the prion protein can be dissociated from the complex with the prion-specific reagent, denatured, and detected using antibodies to the denatured prion protein. The specificity of the method relies upon the ability to separate the pathogenic prion form from the non-pathogenic prion form. Typically, a simple washing of the PrP^{SC}-prion specific reagent complex (as described in U.S. application Ser. No. 10/917,646; U.S. application Ser. No. 11/056,950, U.S. application Ser. No. 11/518,091 and WO2007/030804) is sufficient to remove any non-pathogenic prion protein. However, the present inventors have discovered that a small percentage of samples, particularly blood samples, contain a very high level of non-pathogenic prion protein which may be incompletely removed by simple washing. This incomplete removal of the non-pathogenic prion protein leads to a high background level of signal and can result in a false positive report or obscure a true positive signal.

The present invention provides an improvement to the previously described methods of detection that reduces the background signal that occurs in this small percentage of samples due to unusually high levels of PrP^{C}. The present inventors have found that adding a step of treatment of the complex with a site-specific protease, as further described herein, reduces the background level of signal due to non-pathogenic prion protein that is incompletely removed by simple washing, without significantly affecting the signal from the pathogenic prion protein.

Thus, provided herein are methods for detecting the presence of a pathogenic prion in a sample by (a) contacting the sample with a pathogenic prion-specific reagent under conditions that allow binding of the reagent to the pathogenic prion, if present, to form a first complex; (b) contacting the first complex with a site-specific protease under conditions in which non-pathogenic prion protein is substantially digested by the protease, (c) removing said digested non-pathogenic prions and any unbound sample from the first complex; (d) dissociating the pathogenic prion from the first complex thereby providing dissociated pathogenic prion; (e) contacting the dissociated pathogenic prion with a first anti-prion antibody under conditions that allow binding of the anti-prion antibody to the pathogenic prion to form a second complex; and (1) detecting formation of the second complex, wherein the formation of the second complex is indicative of the presence of the pathogenic prion. In any of the methods described herein, the pathogenic prion-specific reagent is preferably a peptide reagent (as described in U.S. application Ser. Nos. 10/917,646 and 11/056,950) or peptoid reagent (as described in U.S. application Ser. No. 11/518,091).

In certain embodiments, the methods further comprise detecting the second complex with a second (optionally detectably labeled) anti-prion antibody.

In any of the methods described herein, the non-specifically bound non-pathogenic prions can be removed by treating the first complex with a site-specific protease. The site-specific protease is preferably one that does not cleave the prion protein at a site within the octarepeat region. In certain embodiments, the protease used to remove non-pathogenic prion proteins from the first complex comprises trypsin or SV-8. Once the non-pathogenic prions are substantially digested by the site-specific protease, the protease is removed, inactivated or inhibited in order that further protease digestion (e.g., of other protein components) is prevented. Typically, the protease activity can be removed, inactivated or inhibited by additional washing of the first complex, and/or by the addition of a protease inhibitor, or by other methods well known in the art.

In certain embodiments, the step of dissociating the pathogenic prion from the first complex is carried out by exposing the complex to high pH or low pH and, optionally, neutralizing said high pH or said low pH after said dissociating. The dissociated pathogenic prion may be denatured.

In certain embodiments, the pathogenic-prion specific reagent and/or the first anti-prion antibody is bound to a solid support. In certain embodiments, the pathogenic prion-specific reagent is bound to a magnetic bead and/or the first anti-prion antibody is bound to a microtiter plate.

Thus, in one embodiment, the invention provides a method for detecting the presence of a pathogenic prion in a sample suspecting of containing pathogenic and non-pathogenic prions, comprising the steps of:

(a) contacting the sample with a pathogenic prion-specific reagent under conditions that allow binding of said reagent to said pathogenic prion, if present, to form a first complex;

(b) contacting said first complex with a site-specific protease under conditions in which the non-pathogenic prions are substantially digested by the protease;

(c) preventing further cleavage by the site-specific protease;

(d) separating the first complex from any unbound sample and from cleaved non-pathogenic prions;

(e) dissociating said pathogenic prion from said first complex thereby providing dissociated pathogenic prion;

(f) contacting said dissociated pathogenic prion with a first anti-prion antibody under conditions that allow binding of said first anti-prion antibody to said pathogenic prion to form a second complex; and

(g) detecting formation of said second complex by contacting said second complex with a second anti-prion antibody, optionally labeled;

wherein said first anti-prion antibody recognizes a first epitope in said prion protein and said second anti-prion antibody recognizes a second epitope in said prion protein, wherein said first and second epitopes are not the same and are separated by at least one cleavage site for the site-specific protease, and wherein said at least one cleavage site for said site-specific protease is located within said proteinase K resistant core region of said prion protein.

In addition, in any of the methods described herein the sample can be a biological sample, that is, a sample obtained or derived from a living or once-living organism, for example, organs, whole blood, blood fractions, blood components, plasma, platelets, serum, cerebrospinal fluid (CSF), brain tissue, nervous system tissue, muscle tissue, bone marrow, urine, tears, non-nervous system tissue, organs, and/or biopsies or necropsies. In preferred embodiments, the biological sample comprises blood, blood fractions or blood components. The sample may be a non-biological sample.

In another aspect, the present disclosure provides a method of diagnosing a prion-related disease in a subject by detecting the presence of a pathogenic prion in a biological sample from said subject by any of the detection methods described herein.

In another aspect, various kits for detecting the presence of a pathogenic prion in a sample are provided, the kit comprising: one or more reagents that interact preferentially with pathogenic prions (i.e., pathogenic prion-specific reagents); and/or any of the solid supports comprising one or more of these reagents, anti-prion antibodies, and other necessary reagents and, optionally, positive and negative controls. In certain embodiments, the kit also includes a suitable site-specific protease, and optionally protease inhibitor.

These and other embodiments will readily occur to those of skill in the art in light of the disclosure herein.

### BRIEF DESCRIPTION OF THE DRAWINGS

FIG. 1 is a graph depicting ELISA results (RLU) of plasma samples containing PrP^{SC}. Samples treated with trypsin are shown in dark gray (right bars). Samples not treated with trypsin are shown in light gray (left bars).

FIG. 2 is a schematic of the improved method of the present invention. The single line represents the prion protein, the coiled section represents the protease resistant core of the PrP^{SC}, the wavy section represents the more alpha-helical sections of the PrP^{C} and PrP^{SC} isoforms. The boxes indicate the epitope regions recognized by the anti-prion antibodies, the triangles represent the protease cleavage sites.

FIGS. 3A, 3B shows the sequence alignment for several species of prion proteins indicating the octarepeat regions (double underlined), the proteinase resistant core regions (bracketed) and potential trypsin protease cleavage sites (single underline, bold). Trypsin cleaves at the carboxyl side of Lys and Arg residues, except when there is an adjacent Pro at the carboxyl side which hinders the cleavage.

FIGS. 4A, 4B shows the sequence alignment for several species of prion proteins indicating the octarepeat regions (double underlined), the proteinase resistant core regions (bracketed) and potential SV-8 protease cleavage sites (single underline, bold). SV-8 cleaves at Glu and Asp residues.

### DETAILED DESCRIPTION

The practice of the present disclosure will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B.N. Raven Press, New York, N.Y.

All publications, patents and patent applications cited herein are hereby incorporated by reference in their entirety.

### DEFINITIONS

The following select terms will be discussed in the context used herein. Both the plural and singular forms of a term are included regardless of the form discussed.

“Prion,” “prion protein,” “PrP protein,” and “PrP” are used interchangeably to refer to both the pathogenic prion protein form (also referred to as scrapie protein, pathogenic protein form, pathogenic isoform, pathogenic prion and PrP^{C}) and the non-pathogenic prion form (also referred to as cellular protein form, cellular isoform, nonpathogenic isoform, nonpathogenic prion protein, and PrP^{C}), as well as the denatured form and various recombinant forms of the prion protein that may not have either the pathogenic conformation or the normal cellular conformation.

Use of the terms “prion,” “prion protein,” “PrP protein,” “PrP” or “conformational disease protein” is not meant to be limited to polypeptides having the exact sequences to those described herein. It is readily apparent that the terms encompass conformational disease proteins from any of the identified or unidentified species (e.g., human, bovine) or diseases (e.g., Alzheimer's, Parkinson's, etc.). See also, co-owned U.S. Patent Publications 20050118645 and 20060035242 and PCT Publication WO 06/076687, which are incorporated herein by reference in their entireties. One of ordinary skill in the art in view of the teachings of the present disclosure and the art can determine regions corresponding to the sequences disclosed herein in any other prion proteins, using for example, sequence comparison programs (e.g., Basic Local Alignment Search Tool (BLAST)) or identification and alignment of structural features or motifs.

“Pathogenic” means that the protein actually causes the disease, or the protein is associated with the disease and, therefore, is present when the disease is present. Thus, a pathogenic protein, as used herein, is not necessarily a protein that is the specific causative agent of a disease. Pathogenic forms of a protein may or may not be infectious. An example of a pathogenic conformational disease protein is PrP^{Sc}. Accordingly, the term “non-pathogenic” describes a protein that does not normally cause disease or is not normally associated with causing disease. An example of a non-pathogenic conformational disease protein is PrP^{C}.

“Interact” in reference to a reagent (e.g., peptide or peptoid) interacting with a protein, e.g., a protein fragment, means the reagent binds specifically, non-specifically or in some combination of specific and non-specific binding to the prion protein. A reagent is said to “interact preferentially” with a pathogenic prion protein if it binds with greater affinity and/or greater specificity to the pathogenic form than to nonpathogenic isoforms. Thus, a reagent that interacts preferentially with a pathogenic prion protein is also referred to herein as a “pathogenic prion-specific reagent.” In some embodiments, the increased affinity and/or specificity is at least about 2-fold, at least about 5-fold, at least about 10-fold, at least about 50-fold, at least about 100-fold, at least about 500-fold, or at least about 1000-fold. It is to be understood that a preferential interaction does not necessarily require interaction between a specific amino acid or amino acid substitute residues and/or motifs of each peptide. For example, in some embodiments, the reagents interact preferentially with pathogenic isoforms but, nonetheless, can be capable of binding nonpathogenic isoforms at a weak, yet detectable, level (e.g., 10% or less of the binding shown to the polypeptide of interest). Typically, weak binding, or background binding, is readily discernible from the preferential interaction with the compound or polypeptide of interest, e.g., by use of appropriate controls. In general, reagents used in the detection methods described herein bind pathogenic prions in the presence of a 10⁶-fold excess of nonpathogenic forms. Peptide reagents and peptoid reagents that interact preferentially with the pathogenic form of the prion protein are described in detail in U.S. application Ser. No. 10/917,646; U.S. application Ser. No. 11/056,950, U.S. application Ser. No. 11/518,091 and WO2007/030804.

“Affinity” or “binding affinity,” in terms of the reagent interacting with a conformational disease protein, refers to the strength of binding and can be expressed quantitatively as a dissociation constant (K_{d}). Binding affinity can be determined using techniques well known by one of ordinary skill in the art.

“Prion-related disease” refers to a disease caused in whole or in part by a pathogenic prion protein (e.g., PrP^{Sc}), for example, but without limitation, scrapie, bovine spongiform encephalopathies (BSE), mad cow disease, feline spongiform encephalopathies, kuru, Creutzfeldt-Jakob Disease (CJD), new variant Creutzfeldt-Jakob Disease (nvCJD), chronic wasting disease (CWD), Gerstmann-Strassler-Scheinker Disease (GSS), and fatal familial insomnia (FFI).

The term “denature” or “denatured” has the conventional meaning as applied to protein structure and means that the protein has lost its native secondary and tertiary structure. With respect to the pathogenic prion protein, a “denatured” pathogenic prion protein no longer retains the native pathogenic conformation and thus the protein is no longer “pathogenic.” The denatured pathogenic prion protein has a conformation similar or identical to the denatured non-pathogenic prion protein. However, for purposes of clarity herein, the term “denatured pathogenic prion protein” will be used to refer to the pathogenic prion protein that is captured by the reagent as the pathogenic isoform and subsequently denatured.

“Physiologically relevant pH” refers to a pH of about 5.5 to about 8.5; or about 6.0 to about 8.0; or usually about 6.5 to about 7.5.

“Peptide” is used generally to refer to any compound comprising naturally occurring or synthetic polymers of amino acid or amino acid-like molecules, including but not limited to compounds comprising only amino and/or imino molecules. The term “peptide” is used interchangeably with “oligopeptide” and “polypeptide.” No particular size is implied by use of these terms. Included within the definition are, for example, peptides containing one or more analogs of an amino acid (including, for example, unnatural amino acids, etc.), peptides with substituted linkages, as well as other modifications known in the art, both naturally occurring and non-naturally occurring (e.g., synthetic). Thus, synthetic peptides, dimers, multimers (e.g., tandem repeats, multiple antigenic peptide (MAP) forms, linearly-linked peptides), cyclized, branched molecules and the like, are included within the definition.

“Peptoid” is used generally to refer to a peptide mimic that contains at least one, preferably two or more, amino acid substitutes, preferably N-substituted glycines. Peptoids are described in, inter alia, U.S. Pat. No. 5,811,387.

The terms “label,” “labeled,” “detectable label,” and “detectably labeled” refer to a molecule capable of detection, including, but not limited to, radioactive isotopes, fluorescers, luminescers, chemiluminescers, enzymes, enzyme substrates, enzyme cofactors, enzyme inhibitors, chromophores, dyes, metal ions, metal sols, ligands (e.g., biotin or haptens), fluorescent nanoparticles, gold nanoparticles, and the like. The term “fluoresce” refers to a substance or a portion thereof that is capable of exhibiting fluorescence in the detectable range such as a fluorophore. Particular examples of labels that can be used include, but are not limited to fluorescein, rhodamine, dansyl, umbelliferone, Texas red, luminol, acridinium esters, NADPH, beta-galactosidase, horseradish peroxidase, glucose oxidase, alkaline phosphatase and urease. The label can also be an epitope tag (e.g., a His-His tag), an antibody or an amplifiable or otherwise detectable oligonucleotide.

A “pathogenic prion-specific reagent” or “PSR” refers to reagents, generally peptides or peptoids, that interact preferentially with pathogenic prion proteins by which is meant that the PSR binds with greater affinity and/or greater specificity to the pathogenic prion forms than to the non-pathogenic prion forms. The PSRs have other, additional physical characteristics that are fully described in U.S. application Ser. Nos. 10/917,646; 11/056,950; and 11/518,091. Preferred PSRs for use in connection with the methods of the present invention include those described in the above referenced applications, particularly peptide reagents comprising or derived from SEQ ID NO: 12-132, particularly from SEQ ID NO: 66, 67, 68, 72, 81, 96, 97, 98, 107, 108, 119, 120, 121, 122, 123, 124, 125, 126, 127, 14, 35, 36, 37, 40, 50, 51, 77, 89, 100, 101, 109, 110, 111, 112, 113, 114, 115, 116, 117, 118, 128, 129, 130, 131, 132, 56, 57, 65, 82, or 84, of U.S. Ser. No. 10/917,646, filed Aug. 13, 2004, the disclosure of which is incorporated herein by reference, and peptoid reagents comprising or derived from SEQ ID NO: 230, 237, 238, 239, or 240 or compounds I, II, III, N, V, VI, VII, VIII, IX, X, XIa, XIb, XIIa, XIIb, or XIII of U.S. application Ser. No. 11/518,091, filed Sep. 8, 2006, the disclosure of which is incorporated by reference herein.

A “site-specific protease” refers to an enzyme that cleaves peptide bonds (a protease) at one type or a small number of different amino acid residues in a protein. For example, trypsin is a site-specific protease that cleaves only at Lys and Arg residues. The site-specific protease is distinguished from the non-specific proteases like proteinase K (which cleaves at all aliphatic, aromatic and hydrophobic residues) and carboxypeptidase Y (which cleaves all residues sequentially beginning at the carboxy terminal).

“Substantially digested” means that a protein has been cleaved by a protease in at least 90%, preferably 99%, of all available protease cleavage sites. By “available protease cleavage site” is intended those sites having the amino acid sequence recognized as the cleavage site by the protease and that are available for contact with the protease in the conformation of the protein. As an example, protease cleavage sites that occur within the proteinase K resistant core of the prion protein are generally not available to protease digestion when the prion protein is in the PrP^{SC} conformation.

“Octarepeat region” refers to a repeated sequence region that is found close to the N-terminal of the mature prion proteins from all species so far identified. The octarepeat generally contains between 3 and 5, usually 4, copies of an 8 (or 9) amino acid sequence usually written as GQPHGG(G/S)(-/G)W (SEQ ID NO:11). This sequence is highly conserved (although this sequence may vary slightly in some of the repeats) and generally occurs within about residues 58-91. The octarepeat region is usually adjacent to, and N-terminal proximal of, the proteinase K resistant core region.

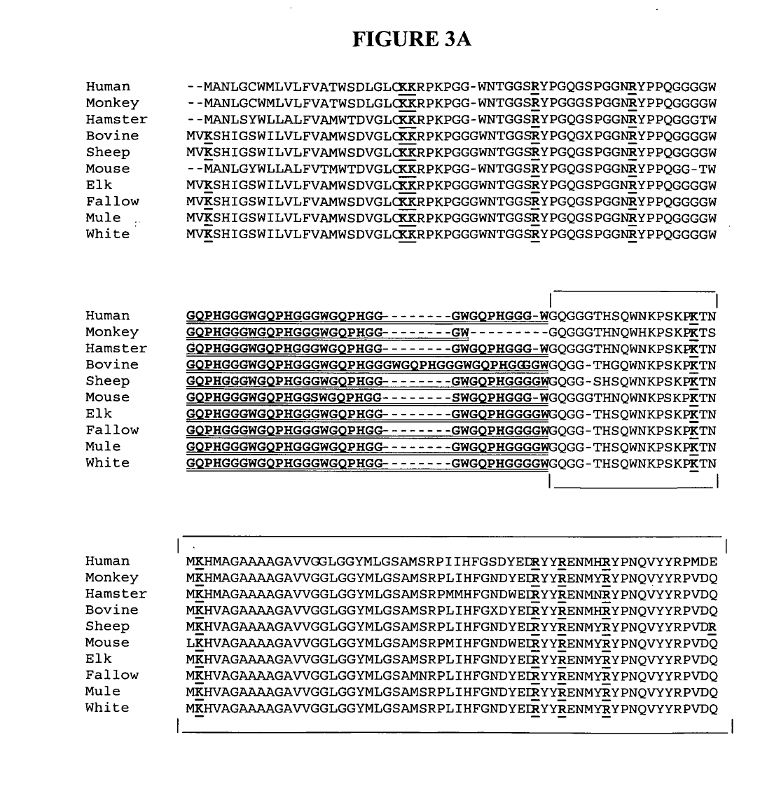

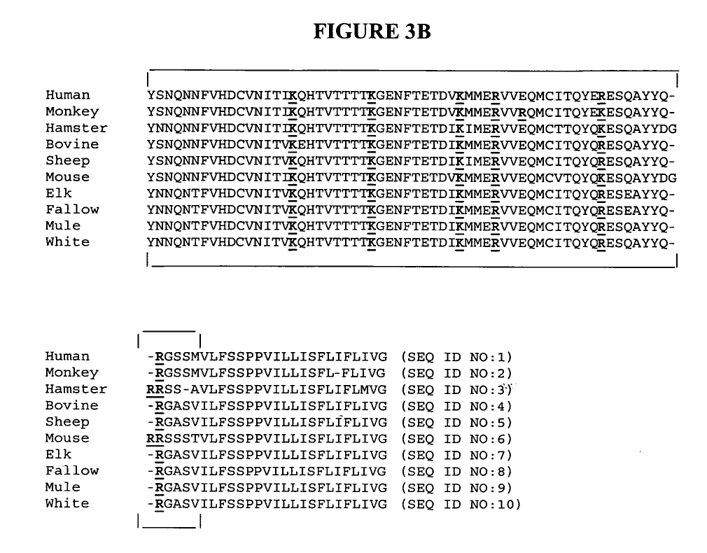

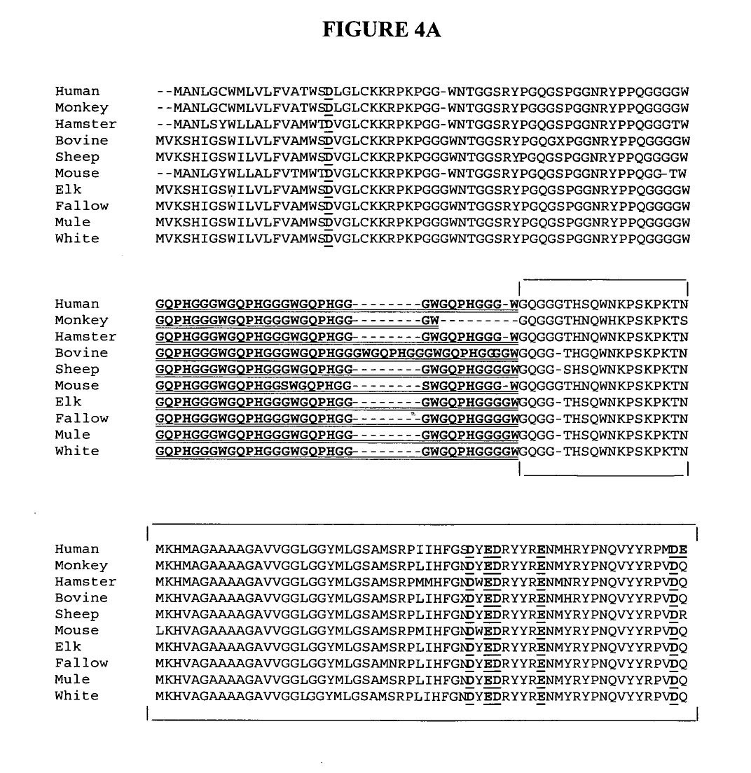

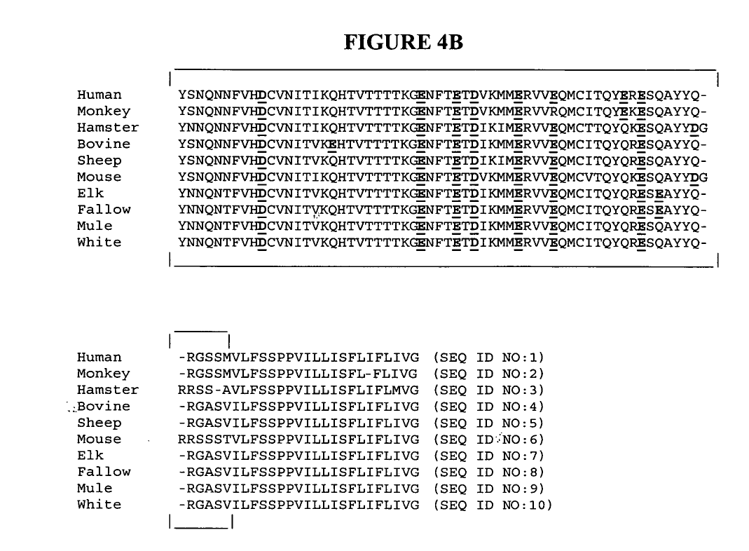

The “proteinase K resistant core” of the prion protein (sometimes called the “protease resistant core”) is defined by the region of the prion protein in the PrP^{SC} conformation that remains after exposure of the PrP^{SC} to proteinase K under condition that are sufficient to substantially digest the prion protein in the PrP^{C} form. In general, for most species of prion protein, the proteinase K resistant core region includes the regions from about amino acid 90 to about amino acid 231. FIGS. 3 and 4 show alignment of prion proteins from 10 different species where the boxed region indicates the proteinase K resistant region.

A “prion-binding reagent” is a reagent that binds to a prion protein in some conformation, e.g., the prion-binding reagent may bind to one or more of a denatured form of the prion protein, the PrP^{C} form (non-pathogenic isoform), or the PrP^{SC} (pathogenic isoform). Some such prion-binding reagents will bind to more than one of these prion protein forms. Thus, the prion-binding reagents include, but are not limited to, the PSRs, which preferentially interact with the pathogenic prion. Prion-binding reagents specifically binds to prions in any form. Prion-binding reagents have been described and include, for example, anti-prion antibodies (described, inter alia, in Peretz et al. 1997 J. Mol. Biol. 273: 614; Peretz et al. 2001 Nature 412: 739; Williamson et al. 1998 J. Virol. 72: 9413; Polymenidou et al. The Lancet 2005 4:805; U.S. Pat. No. 4,806,627; U.S. Pat. No. 6,765,088; and U.S. Pat. No. 6,537,548), motif-grafted hybrid polypeptides (see, WO03/085086), certain cationic or anionic polymers (see, WO03/073106), certain peptides that are “propagation catalysts” (see, WO02/097444), prion specific peptide reagents (see, for example, WO2006/076687 and US20060035242) and plasminogen. In all of the methods utilizing a prion-binding reagent, preferred prion-binding reagents are anti-prion antibodies.

An “epitope” is a site on an antigen to which specific B cells and/or T cells respond, rendering the molecule including such an epitope capable of eliciting an immunological reaction or capable of reacting with antibodies present in a biological sample. The term is also used interchangeably with “antigenic determinant” or “antigenic determinant site.” An epitope can comprise 3 or more amino acids in a spatial conformation unique to the epitope. Generally, an epitope consists of at least 5 such amino acids and, more usually, consists of at least 8-10 such amino acids. Methods of determining spatial conformation of amino acids are known in the art and include, for example, x-ray crystallography and 2-dimensional nuclear magnetic resonance. Furthermore, the identification of epitopes in a given protein is readily accomplished using techniques well known in the art, such as by the use of hydrophobicity studies and by site-directed serology. See, also, Geysen et al., Proc. Natl. Acad. Sci. USA (1984) 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). Antibodies that recognize the same epitope can be identified in a simple immunoassay showing the ability of one antibody to block the binding of another antibody to a target antigen.

### General Overview

The discovery of reagents (peptide and peptoid) that preferentially interact with pathogenic prion proteins has allowed for the detection of PrP^{SC} in biological samples that contain orders of magnitude more PrP^{C}. See, U.S. Patent Publications 20050118645 and 20060035242; PCT Publication WO 06/076687. The methods described do not require the use proteinase K to pre-treat the sample. As described in these publications, one such detection assay involves using magnetic beads coated with pathogenic prion-specific reagents and contacting the beads with samples suspected of containing PrP^{C} and PrP^{SC} under conditions that allow binding of the pathogenic form to the reagent-containing beads to form a complex. After capture of PrP^{SC} and washing, PrP^{SC} is dissociated from the beads, typically by denaturation by exposure to high or low pH, neutralized, and detected by simple ELISA or preferably by a sandwich ELISA (Enzyme-Linked Immunosorbent Assay). This protocol detects PrP^{SC} in human plasma samples spiked with a million-fold dilution of 10% brain homogenate from humans known to have died from prion disease.

However, the present inventors have found that PrP^{C} can bind non-specifically to reagent-coated beads and may interfere with detection of PrP^{SC} using these methods if not completely removed from the beads. Typically, the non-specifically bound PrP^{C} can be removed from the beads by a simple washing. However, the present inventors have also found that the amount of PrP^{C} naturally varies greatly between different samples and, when present in significant amounts, may not be removed by a simple washing (or repeated washings) and can interfere with detection of PrP^{Sc} by indicating a false positive result or by masking a true positive signal because of the high background.

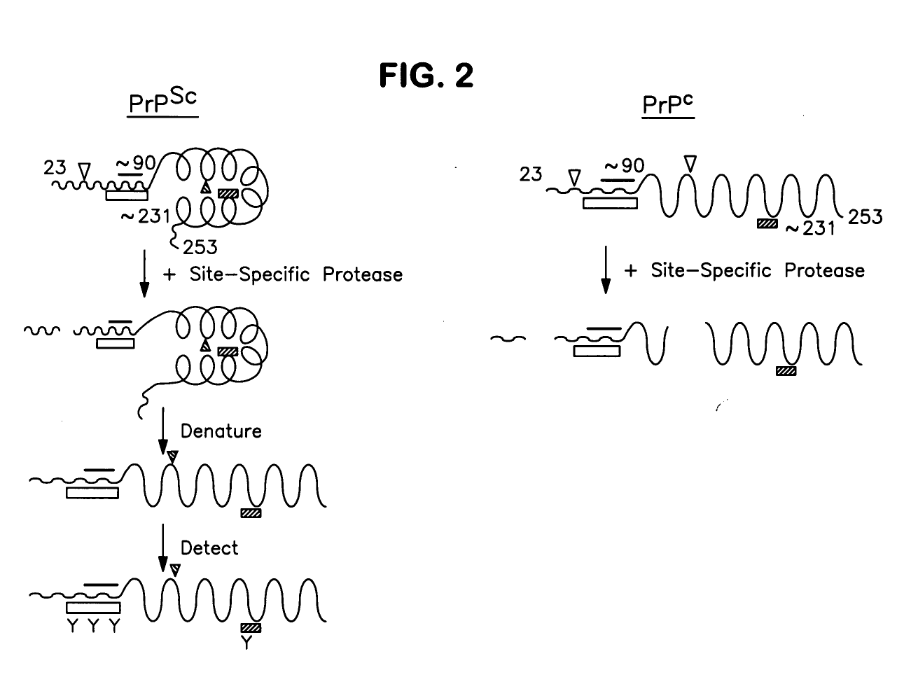

Thus, the methods described herein relate to improvements that can increase specificity of detection of PrP^{SC} captured with pathogenic prion-specific reagents by removing non-specifically bound PrP^{C} prior to ELISA detection of PrP^{SC}. In a preferred embodiment, PrP^{C} is removed by treating the pathogenic prion-specific reagent-PrP^{SC} complex (that may also include non-specifically bound PrP^{C}) with a site-specific protease that cleaves the PrP^{C} within the 90-231 residue region (corresponding to the PK resistant core in the PrP^{SC} form) but not within the octarepeat region. The site-specific protease is selected such that the PrP^{SC} isoform is not cleaved by the protease within the PK resistant core region (because the PrP^{SC} structure in that region makes the potential cleavage sites unavailable), within the octarepeat region or between these two regions. After treatment with the site-specific protease, PrP^{SC} can be detected in a sandwich ELISA technique that uses two different anti-prion antibodies, one that recognizes an epitope in the octarepeat and one that recognizes an epitope that is in the PK resistant core region after (that is, carboxy terminal proximal to) at least one recognition site for the site-specific protease in the PK resistant core region. FIG. 2 provides a schematic that indicates the relationship of the potential cleavage sites, both available (open triangle) and unavailable (striped triangle) in the prion isoforms for the site-specific protease, the epitopes recognized by the anti-prion antibodies used in the ELISA (boxes), the proteinase resistant core region of the PrP^{SC} (coiled line), the alpha-helical regions (wavy lines), and the octarepeat sequence (solid bar).

Accordingly, the methods described herein allow for the improved detection of pathogenic prions in a sample using peptide reagents and peptoid reagents that interact preferentially with pathogenic prion forms combined with an ELISA technique.

The present invention thus provides a method for detecting the presence of a pathogenic prion in a sample suspected of containing pathogenic and non-pathogenic prions, comprising

(a) contacting the sample with a pathogenic prion-specific reagent under conditions that allow binding of said reagent to said pathogenic prion, if present, to form a first complex;

(b) contacting said first complex with a site-specific protease under conditions in which the non-pathogenic prions are substantially digested by the protease;

(c) preventing further cleavage by the site-specific protease;

(d) separating the first complex from any unbound sample and from digested non-pathogenic prions;

(e) dissociating said pathogenic prion from said first complex thereby providing dissociated pathogenic prion;

(f) contacting said dissociated pathogenic prion with a first anti-prion antibody under conditions that allow binding of said first anti-prion antibody to said pathogenic prion to form a second complex; and

(g) detecting formation of said second complex by contacting said second complex with a second anti-prion antibody, optionally labeled;

wherein said first anti-prion antibody recognizes a first epitope in said prion protein and said second anti-prion antibody recognizes a second epitope in said prion protein, wherein said first and second epitopes are not the same and are separated by at least one cleavage site for the site-specific protease, and wherein said at least one cleavage site for said site-specific protease is located within said proteinase K resistant core region of said prion protein.

### Pathogenic Prion-Specific Reagents

The assays described herein utilize reagents that preferentially interact with pathogenic prion forms. In a particularly preferred embodiment, the pathogenic prion-specific reagents are peptide reagents or peptoid reagents as described in U.S. Patent Publications 20050118645 and 20060035242; and PCT/US2006/035226 (WO2007/030804). Preferred PSRs for use in connection with the methods of the present invention include those described in the above referenced applications, particularly peptide reagents comprising or derived from SEQ ID NO: 12-132, particularly from SEQ ID NO: 66, 67, 68, 72, 81, 96, 97, 98, 107, 108, 119, 120, 121, 122, 123, 124, 125, 126, 127, 14, 35, 36, 37, 40, 50, 51, 77, 89, 100, 101, 109, 110, 111, 112, 113, 114, 115, 116, 117, 118, 128, 129, 130, 131, 132, 56, 57, 65, 82, or 84, of U.S. Ser. No. 10/917,646, filed Aug. 13, 2004, the disclosure of which is incorporated herein by reference, and peptoid reagents comprising or derived from SEQ ID NO: SEQ ID NO: 230, 237, 238, 239, or 240 or compounds I, II, III, IV, V, VI, VII, VIII, IX, X, XIa, XIb, XIIa, XIIb, or XIII of U.S. application Ser. No. 11/518,091, filed Sep. 8, 2006, the disclosure of which is incorporated by reference herein.

The pathogenic prion-specific reagents used in the methods described herein are preferably attached to a solid support. These reagents can be provided on a solid support prior to contacting the sample, or the pathogenic prion-specific reagent can be adapted for binding to the solid support after contacting the sample and binding to any pathogenic prion therein (e.g., by using a biotinylated reagent and a solid support comprising an avidin or streptavidin).

Suitable solid supports include any material that is an insoluble matrix and has a rigid or semi-rigid surface to which the pathogenic-prion specific reagent can be linked or attached. Exemplary solid supports include, but are not limited to, substrates such as nitrocellulose, polyvinylchloride, polypropylene, polystyrene, latex, polycarbonate, nylon, dextran, chitin, sand, silica, pumice, agarose, cellulose, glass, metal, polyacrylamide, silicon, rubber, polysaccharides, polyvinyl fluoride; diazotized paper, activated beads, magnetically responsive beads, and any materials commonly used for solid phase synthesis, affinity separations, purifications, hybridization reactions, immunoassays and other such applications. The support can be particulate or can be in the form of a continuous surface and includes membranes, mesh, plates, pellets, slides, disks, capillaries, hollow fibers, needles, pins, chips, solid fibers, gels (e.g. silica gels) and beads, (e.g., pore-glass beads, silica gels, polystyrene beads optionally cross-linked with divinylbenzene, grafted co-poly beads, polyacrylamide beads, latex beads, dimethylacrylamide beads optionally crosslinked with N—N′-bis-acryloylethylenediamine, iron oxide magnetic beads, and glass particles coated with a hydrophobic polymer. In a preferred embodiment, the pathogenic prion-specific reagent is attached to a magnetic bead.

Pathogenic prion-specific reagents as described herein can be readily coupled to the solid support using standard techniques. Immobilization to the support may be enhanced by first coupling the reagent to a protein (e.g., when the protein has better solid phase-binding properties). Suitable coupling proteins include, but are not limited to, macromolecules such as serum albumins including bovine serum albumin (BSA), keyhole limpet hemocyanin, immunoglobulin molecules, thyroglobuline, ovalbumin, and other proteins well known to those skilled in the art. Other reagents that can be used to bind molecules to the support include polysaccharides, polylactic acids, polyglycolic acids, polymeric amino acids, amino acid copolymers, and the like. Such molecules and methods of coupling these molecules to proteins, are well known to those of ordinary skill in the art. See, e.g., Brinkley, M. A., (1992) Bioconjugate Chem., 3:2-13; Hashida et al. (1984) J. Appl. Biochem., 6:56-63; and Anjaneyulu and Staros (1987) International J. of Peptide and Protein Res. 30:117-124.

If desired, the pathogenic prion-specific reagent can readily be functionalized to create styrene or acrylate moieties, thus enabling the incorporation of the molecules into polystyrene, polyacrylate or other polymers such as polyimide, polyacrylamide, polyethylene, polyvinyl, polydiacetylene, polyphenylene-vinylene, polypeptide, polysaccharide, polysulfone, polypyrrole, polyimidazole, polythiophene, polyether, epoxies, silica glass, silica gel, siloxane, polyphosphate, hydrogel, agarose, cellulose and the like.

The pathogenic prion-specific reagents (PSR) can also be attached to the solid support through the interaction of a binding pair of molecules. Such binding pairs are well known and examples are described elsewhere herein. One member of the binding pair is coupled by techniques described above to the solid support and the other member of the binding pair is attached to the PSR (before, during, or after synthesis). The PSR thus modified can be contacted with the sample and interaction with the pathogenic prion, if present, can occur in solution, after which the solid support can be contacted with the peptide reagent (or peptide-prion complex). Preferred binding pairs for this embodiment include biotin and avidin, and biotin and streptavidin.

Thus, in the methods described herein, pathogenic prion forms in a sample are captured using a PSR (e.g., peptide reagent or peptoid reagent) that preferentially binds to the pathogenic form.

### Site-Specific Protease

Site-specific proteases that are useful in the present invention are proteases that cleave peptide bonds of specific, discrete amino acid residues. Generally, the site-specific proteases will cleave a protein at one type or a small number of specific amino acid residues thus allowing predictability in the cleavage of the prion protein. Examples of such site-specific proteases are: trypsin which is a site-specific protease that cleaves on the carboxyl side of Arg or Lys residues and SV-8 which is a site-specific protease from Staphyloccocus aureas that cleaves on the carboxyl side of Asp or Glu residues. Both trypsin and SV-8 are commercially available from various suppliers (e.g., Pierce Rockford Ill.). Other such site-specific proteases can be readily selected by one of ordinary skill in the art based on the description herein. In addition, to be useful in the present method, there must be at least one cleavage site for the site-specific protease in the prion protein in the region between the epitopes recognized by the two antibodies used for the ELISA and the at least one protease cleavage sequence will be within the PK resistant core region (approximately amino acids 90-231 of the prion protein). This site will be cleaved by the site-specific protease only when the prion protein is in the PrP^{C} form and not when the prion protein is in the PrP^{SC} form. Additionally, there will be no potential cleavage sites available for cleavage in the PrP^{SC} isoform in the region between the two epitopes. Preferably at least one of the epitopes will be in the PK resistant core region of the prion protein. Preferably, the other epitope will be in the octarepeat region of the prion protein. Preferably, the site-specific protease does not cleave at a site within the octarepeat region of the prion protein. The core repeated sequence of the octarepeat region is GQPHGG(G/S)(-/G)W (SEQ ID NO: 11), which can vary slightly in prions from different species (See FIGS. 3 and 4 for the sequences of 10 different prion proteins showing the octarepeat sequences.) FIG. 2 shows a schematic representation of the PrP^{C} and PrP^{SC} forms showing the octarepeat regions, exemplary epitope sites and exemplary site-specific protease cleavage sites. The site-specific protease will cleave the PrP^{C} form in at least one site between the epitopes recognized by the anti-prion antibodies used in the ELISA. Thus, the PrP^{C} form will not be detected in the ELISA. The PrP^{SC} form, however, will not be cleaved by the site-specific protease in the region between the two epitopes because the conformation of this isoform makes the sites unavailable for protease cleavage. Thus, the PrP^{SC} will be detectable in the ELISA.

### Protease Treatment

As described above, the complex formed by specific interaction of the pathogenic prion-specific reagent and pathogenic prion may also include non-specifically bound PrP^{C}, particularly when the sample naturally contains high levels of PrP^{C}. Proteinase K has been used in other settings to digest the PrP^{C} form, leaving the more resistant PrP^{SC} form. However, PrP^{SC} is not completely resistant to proteolysis if high concentrations of proteinase K and/or prolonged exposure times are used as shown by the fact that PK treatment reduces infectivity of the pathogenic form. See, McKinley et al. Cell, Vol. 35, 5 7-62, 1983. In addition, some conformers of the PrP^{SC} have been shown to be more sensitive to proteinase K and such treatment might reduce the sensitivity of detection (Safar et al. (1998) Nature Med. 4:1157)

Therefore, proteinase K treatment must be carefully controlled in order to provide complete cleavage of the PrP^{C} form but leave the resistant core of the PrP^{SC} form intact. Too little proteinase K digestion will leave residual PrP^{C} form which will yield a false positive in the detection phase and too much proteinase K digestion will cleave the PrP^{SC} resistant core making it undetectable in the detection phase. In addition, although the particular PK digestion site(s) of PrP^{SC} vary since the pathogenic form can adopt multiple conformations, PK digestion of PrP^{SC} invariably removes the N-terminal amino acids from about residue 23 to 90 (the mature prion protein begins at amino acid 23). This N-terminal region has sequences, particularly the octarepeat sequence, which can be important epitopes for anti-prion antibodies. Thus, PK digestion of the PrP^{SC} may reduce or eliminate the binding of anti-prion antibodies directed against epitopes in this region. See, Telling et al. Science Vol. 274. pp. 2079-2082, 1996).

The first complex comprising the PSR and the pathogenic prion is contacted with the selected site-specific protease under conditions in which any non-pathogenic prion protein would be substantially digested. One of ordinary skill in the art is competent to determine the appropriate conditions. Conditions of substantial digestion can readily be determined by tests using recombinant PrP. For trypsin as the site-specific protease, typically a trypsin concentration of 50 μg/ml for 1 hour at 37° C. is adequate.

The site-specific protease can be contacted with the first complex immediately after the first complex is formed, or the first complex can be separated from the unbound sample, optionally washed, and then contacted with the site-specific protease.

Following substantial digestion of the non-pathogenic prion protein, the site-specific protease must be removed, inactivated or inhibited in order to prevent any further protease digestion, for instance, of the anti-prion antibodies that will be used for detection. The protease can be removed by simple or repeated washing of the first complex, particularly when the first complex is on a solid support. The protease may also be inhibited by the addition of one or more protease inhibitors. Protease inhibitors are well known in the art and include phenylmethylsulfonyl fluoride (PMSF), aprotinin, diisopropylfluorophosphate (DFP), and 1-chloro-3-tosylamido-4-phenyl-2-butanone (TLCK), among others. Alternately, some proteases are available in an immobilized form (e.g., in an agarose matrix) which can be readily removed from the reaction by conventional means (e.g., centrifugation, filtration, etc.). Typically, PMSF at 1-2 mM will be used to stop the protease digestion.

By removing non-specifically bound PrP^{C} via selective cleavage, the methods described herein increases the specificity and reproducibility of pathogenic prion ELISAs that utilize reagents that preferentially bind to pathogenic prion forms. Following removal of PrP^{C}, anti-prion antibodies, including those that recognize epitopes at the N-terminal end of denatured PrP^{SC}, can be used in ELISAs for detection of pathogenic prions captured by the pathogenic-prion specific reagent.

### ELISAs

Following protease treatment to remove non-pathogenic prions and washing steps, the pathogenic prion protein is dissociated from the pathogenic prion-specific reagent as described in PCT Publication WO 2006/076687 and detected in a number of ELISA formats, described therein and below. The pathogenic prion is typically denatured in the process of dissociation from the pathogenic prion-specific reagent, although not necessarily so. Denaturation of the captured PrP^{SC} before performing the ELISA is preferable, as the majority of high affinity anti-prion antibodies bind the denatured form of PrP and many anti-prion antibodies that bind to the denatured PrP are known and commercially available.

The dissociation and denaturation of the pathogenic prion can be accomplished using high concentrations of chaotropic agents, e.g., 3M to 6M of a guanidinium salt such as guanidinium thiocyanate or guanidinium HCl. The chaotropic agent must be removed or diluted before the ELISA is carried out because it will interfere with the binding of the anti-prion antibodies used in the ELISA. This results in additional washing steps or generation of large sample volumes, both of which are undesirable for rapid, high-throughput assays.

Alternatively, dissociation of the pathogenic prion protein from the reagent can be accomplished using high or low pH. The pathogenic prion protein is readily dissociated from the reagent and denatured by adding components that increase the pH to above 12 (e.g., NaOH) or to below 2 (e.g., H₃PO₄). Moreover, the pH can be easily readjusted to neutral by addition of small volumes of suitable acid or base, thus allowing the use directly in the ELISA without any additional washes and without increasing the sample volumes significantly. The use of high or low pH treatment for denaturing the captured pathogenic prion protein (i.e., the pathogenic prion in the first complex) is described in more detail in PCT/US2006/001437 and U.S. application Ser. No. 11/518,091, the disclosures of which are incorporated herein in their entireties.

Antibodies, modified antibodies and other reagents, that bind to prions, particularly to PrP^{C} or to the denatured PrP, have been described and some of these are available commercially (see, e.g., anti-prion antibodies described in Peretz et al. 1997 J. Mol. Biol. 273: 614; Peretz et al. 2001 Nature 412:739; Williamson et al. 1998 J. Virol. 72:9413; Polymenidou et al. 2005 Lancet 4:805; U.S. Pat. No. 6,765,088. Some of these and others are available commercially from, inter alia, InPro Biotechnology, South San Francisco, Calif., Cayman Chemicals, Ann Arbor Mich.; Prionics AG, Zurich; also see, WO 03/085086 for description of modified antibodies). Suitable antibodies for use in the method include without limitation 3F4 (U.S. Pat. No. 4,806,627), D18 (Peretz et al. J. Mol. Biol. 1997 273:614), D13 (Peretz 1997, supra), 6H4 (Liu et al. J. Histochem. Cytochem. 2003 51:1065), MAB5242 (Chemicon), 7D9 (Kascsak et al. 1987 J. Virol. 61:3688), BDI115 (Biodesign International), SAF32, SAF53, SAF83, SAF84 (SAF antibodies available from SPI Bio, France), 19B10 (WO2004/4033628), 7VC (WO2004/4033628), 12F10 (SPI Bio), PRI308 (SPI Bio), 34C9 (Prionics AG), Fab HuM-P (Peretz et al. Nature 2001 412:739), POM 1 through POM 19 (Polymenidou et al. 2005, supra), particularly POM2 which recognizes an epitope in the octarepeat region, Fab HuM-R1 (Peretz 1997, supra), and Fab HuM-R72 (Peretz 1997, supra). Other anti-prion antibodies can readily be generated by methods that are well-known in the art.

Preferred anti-prion antibodies will be ones that bind to a denatured form of the pathogenic prion. Particularly preferred first anti-prion antibodies will be ones that recognize epitopes at the N-terminal region (e.g., octarepeat region) of the prion protein. Examples of such antibodies are SAF-32, POM2, POM11, POM12, POM14, 3B5, 4F2, 13F10, SAF-15, SAF-31, SAF-32, SAF-33, SAF-34, SAF-35 and SAF-37. (See, e.g., Polymenidou et al. (2005) Lancet Neural. 4:805-814; Krasemann et al. (1996) Mol. Medicine. 2:725-734; Feraudet, et al. (2005) J. Biol. Chem. 280:11247-11258; U.S. Pat. No. 7,097,997 B1.) Preferred second anti-prion antibodies will be ones that recognize epitopes within the proteinase K resistant core region, for example the 3F4 antibody, which recognizes an epitope at about amino acids 109-112, POM17 or POM19. Alternatively, the first anti-prion antibodies can be selected from a group of antibodies that recognize epitopes within the proteinase K resistant core and the second anti-prion antibodies will recognize epitopes at the N-terminal region, particularly within the octarepeat region.

One of skill in the art will appreciate from the disclosure herein that the first and second anti-prion antibodies are selected such that they recognize epitopes that flank a cleavage site for the site-specific protease in the proteinase K resistant core region. In this way, following digestion with the site-specific protease, the epitopes recognized by the first and second anti-prion antibodies will be present on different fragments of the PrP^{C} (and so will not be capable of detection in the ELISA) but these epitopes will be present on a single fragment of the PrP^{SC} (and so will be detectable in the ELISA);

Some anti-prion antibodies are specific for prion protein from one or a limited number of animal species, others are capable of binding prion proteins from many animal species. It will be apparent to choose suitable anti-prion antibodies based upon the samples to be analyzed and the purpose of the testing.

In one embodiment, the pathogenic prion-specific reagent is provided on a solid support, preferably a magnetic bead, more preferably a polystyrene/iron oxide bead.

Methods of attaching a peptide or peptoid reagent on a solid support are conventional in the art and are described elsewhere herein and include well-known methods of attaching proteins and peptides to various solid surfaces.

Typically, the method is carried out in the wells of a microtiter plate or in small volume plastic tubes, but any convenient container will be suitable. The sample is generally a liquid sample or suspension and may be added to the reaction container before or after the pathogenic prion-specific reagent. As noted above, once the first complex is established, non-specifically bound PrP^{C} is removed along with any unbound sample material (that is, any components of the sample that have not bound to the pathogenic prion-specific reagent, including any unbound pathogenic prion protein).

As described above, following the removal of unbound sample materials, removal of any non-specifically bound PrP^{C} and any optional washes, the bound pathogenic prion proteins are dissociated from the first complex. This dissociation can be accomplished in a number of ways. In one embodiment, a chaotropic agent, preferably a guanidinium compound, e.g., guanidinium thiocyanate or guanidinium hydrochloride, is added to a concentration of between 3M and 6M. Addition of the chaotropic agent in these concentrations causes the pathogenic prion protein to dissociate from the reagent and also causes the pathogenic prion protein to denature.

In another embodiment, the dissociation is accomplished by either raising the pH to 12 or above (“high pH”) or lowering the pH to 2 or below (“low pH”). Details of the pH dissociation/denaturation technique are described in PCT/US2006/001437 and U.S. application Ser. No. 11/518,091. Exposure of the first complex to either high or low pH results in the dissociation of the pathogenic prion protein from the reagent and causes the pathogenic prion protein to denature. In this embodiment, exposure of the first complex to high pH is preferred. A pH of between 12.0 and 13.0 is generally sufficient; preferably, a pH of between 12.5 and 13.0 is used; more preferably, a pH of 12.7 to 12.9; most preferably a pH of 12.9. Alternatively, exposure of the first complex to a low pH can be used to dissociate and denature the pathogenic prion protein from the reagent. For this alternative, a pH of between 1.0 and 2.0 is sufficient. Exposure of the first complex to either a high pH or a low pH is carried out for only a short time e.g. 60 minutes, preferably for no more than 15 minutes, more preferably for no more than 10 minutes. Longer exposures than this can result in significant deterioration of the structure of the pathogenic prion protein such that epitopes recognized by anti-prion antibodies used in the detection steps are destroyed. After exposure for sufficient time to dissociate the pathogenic prion protein, the pH can be readily readjusted to neutral (that is, pH of between about 7.0 and 7.5) by addition of either an acidic reagent (if high pH dissociation conditions are used) or a basic reagent (if low pH dissociation conditions are used). One of ordinary skill in the art can readily determine appropriate protocols and examples are described herein.

In general, to effect a high pH dissociation condition, addition of NaOH to a concentration of about 0.05 N to about 0.2 N is sufficient. Preferably, NaOH is added to a concentration of between 0.05 N to 0.15 N; more preferably, 0.1 N NaOH is used. Once the dissociation of the pathogenic prion from the pathogenic prion-specific reagent is accomplished, the pH can be readjusted to neutral (that is, between about 7.0 and 7.5) by addition of suitable amounts of an acidic solution, e.g., phosphoric acid, sodium phosphate monobasic.

In general, to effect a low pH dissociation condition, addition of H₃PO₄ to a concentration of about 0.2 M to about 0.7 M is sufficient. Preferably, H₃PO₄ is added to a concentration of between 0.3 M and 0.6 M; more preferably, 0.5 M H₃PO₄ is used. Once the dissociation of the pathogenic prion from the reagent is accomplished, the pH can be readjusted to neutral (that is, between about 7.0 and 7.5) by addition of suitable amounts of a basic solution, e.g., NaOH or KOH.

The dissociated pathogenic prion protein is then separated from the solid support comprising the pathogenic prion-specific reagent. This separation can be accomplished in similar fashion to the removal of the unbound sample materials described above except that the portion containing the unbound materials (now the dissociated pathogenic prion protein) is retained and the solid support material portion is discarded.

The dissociated pathogenic prion protein can be detected using anti-prion antibodies. A number of anti-prion antibodies have been described and many are commercially available, for example, Fab D18 (Peretz et al. (2001) Nature 412:739-743), 3F4 (available from Sigma Chemical St Louis Mo.; also, See, U.S. Pat. No. 4,806,627), SAF-32 (Cayman Chemical, Ann Arbor Mich.), 6H4 (Prionic AG, Switzerland; also, See U.S. Pat. No. 6,765,088), POMs 1 through 19 (Polymenidou et al. The Lancet 2005 4:805) and others described above and well-known in the art. The dissociated pathogenic prion proteins are preferably detected in an ELISA type assay, either as a direct ELISA or an antibody Sandwich ELISA type assay, which are described more fully below. Although the term “ELISA” is used to describe the detection with anti-prion antibodies, the assay is not limited to ones in which the antibodies are “enzyme-linked.” The detection antibodies can be labeled with any of the detectable labels described herein and well-known in the immunoassay art.

In a preferred embodiment of the method, the dissociated pathogenic prion proteins are detected using an antibody sandwich type ELISA. In this embodiment, the dissociated prion protein is “recaptured” on a second solid support comprising a first anti-prion antibody. The second solid support with the recaptured prion protein, is optionally washed to remove any unbound materials, and then contacted with a second anti-prion antibody under conditions that allow the second anti-prion antibody to bind to the recaptured prion protein.

In this embodiment, the first solid support is preferably a magnetic bead; the second solid support is preferably a microtiter plate or a magnetic bead; the first and second anti-prion antibodies are preferably different antibodies; the first and second antibodies preferably bind to denatured prion protein; preferably, at least one of the first or second anti-prion antibodies recognizes an epitope at the octarepeat region of the prion protein. In some embodiments, the second anti-prion antibody is detectably labeled; in further embodiments, the second anti-prion antibody is enzyme labeled.

Any of the detection methods for a pathogenic prion described hereinabove can be used in a method to diagnose a prion-related disease in any sample.

For use in the methods described herein, the sample can be anything known to, or suspected of, containing a pathogenic prion protein. The sample can be a biological sample (that is, a sample prepared from a living or once-living organism) or a non-biological sample. Suitable biological samples include, but are not limited to, organs, whole blood, blood fractions, blood components, plasma, platelets, serum, cerebrospinal fluid (CSF), brain tissue, nervous system tissue, muscle tissue, bone marrow, urine, tears, non-nervous system tissue, organs, and/or biopsies or necropsies. Preferred biological samples include whole blood, blood products, plasma, platelets and red blood cells.

Suitable controls can also be used in the assays described herein. For instance, a negative control of PrP^{C} can be used in the assays. A positive control of PrP^{Sc} (or PrPres) could also be used in the assays. Such controls can optionally be detectably labeled.

### Kits

The above-described assay reagents, including the pathogenic prion-specific reagents, site-specific proteases, protease inhibitors, denaturing agents, anti-prion antibodies, etc., can be provided in kits, with suitable instructions and other necessary reagents, in order to conduct detection assays as described above. Where the pathogenic prion-specific reagent is employed on a solid support, the kit may additionally or alternatively comprise such reagents on one or more solid supports. The kit may further contain suitable positive and negative controls, as described above. The kit can also contain, depending on the particular detection assay used, suitable labels and other packaged reagents and materials (i.e., wash buffers and the like).

### EXAMPLES

Below are examples of specific embodiments for carrying out the present disclosure. The examples are offered for illustrative purposes only, and are not intended to limit the scope of the present disclosure in any way.

Efforts have been made to ensure accuracy with respect to numbers used (e.g., amounts, temperatures, etc.), but some experimental error and deviation should, of course, be allowed for.

### Example 1

### Protease Treatment of Human Plasma Samples Containing PrP^{C}

To test whether Trypsin digests PrP^{C} sufficiently to render it undetectable in our ELISA, human plasma (104) containing 10 ng/mL of PrP^{C} was treated with increasing concentrations of Trypsin as shown in Table 1. Trypsin digestion was stopped by adding the protease inhibitor phenylmethylsulfonyl fluoride (PMSF) at 1-2 mM, and samples were tested for the presence of PrP^{C} by a sandwich ELISA using 3F4 (obtained from Signet) as capture antibody and detection with POM2 antibody (See Polymenidou et al, supra) conjugated to Alkaline Phosphatase (AP) using a chemiluminescence substrate for light detection. Measurement units are defined in relative light units (RLU). As expected, Trypsin digests PrP^{C} and detection is abolished. At Trypsin concentration of 400 μg/mL and above, the signal for PrP^{C} dropped to background level, suggesting that the PrP^{C} was completely digested.

TABLE 1

| Trypsin | Plasma | | |
|---|---|---|---|
| (μg/mL) | Ave (RLU) | SD | % cv |
| 0 | 195.4 | 3.1 | 1.6 |
| 100 | 70.5 | 1.6 | 2.3 |
| 200 | 10.0 | 0.3 | 2.6 |
| 300 | 8.5 | 0.5 | 6.2 |
| 400 | 1.9 | 0.1 | 5.0 |
| 500 | 1.2 | 0.4 | 29.8 |

### Example 2

### Protease Treatment of Human Plasma Samples Containing PrP^{C} and PrP^{Sc}

The objective of the next set of experiments was to study the effectiveness of Trypsin in eliminating PrP^{C} contaminant in a pull-down assay of PrP^{SC} using magnetic beads coated with a PSR as described in WO2007/030804. Magnetic beads were coated with a peptoid reagent and incubated with different preparations of plasma, with and without addition of 10 nL/mL of vCJD 10% brain homogenate. 10% vCJD brain homogenate was spiked into 200 μL of different normal (i.e., non-vCJD) human plasmas at final concentration of 10 nL/mL. For this experiment two groups of plasma were tested; the first group (N91835, Pools 7-11) had low background (0.8-2.9 relative light units, RLU) indicating a low level of PrP^{C}, and the second group had high levels of PrP^{C} resulting in a much higher background signal (15-114 RLU). These background signals had been determined in previous experiments and reflect the levels of PrP^{C} that were attached to the beads nonspecifically. Adding 10 mL/ml of vCJD 10% brain homogenate resulted in an increase of RLU due to the detection of PrP^{SC} in addition to the PrP^{C}. After mixing for 1 hour at 37° C., beads were collected by magnet, and washed. The beads were treated with Trypsin at 50 μg/ml for 1 hr at 37° C. and proteolysis was stopped by adding 1 mM PMSF for 10 min. at RT. Beads were washed again and the PrP^{SC} in the complex was denatured with NaOH as described in PCT/US06/001437 and WO2007/030804. PrP levels were monitored with ELISA as described in Example 1. The results are shown in Table 2.

To determine the analytical sensitivity, the ratio of vCJD spiked into normal plasma was calculated (S/CO). To achieve statistical confidence of 99.7% the cutoff was defined as the average of the normal plasma plus three standard deviations, and ratios greater than 1.00 were considered positive. For example, for plasma N91835, the cutoff was calculated to be 3 (2.1+3×0.3), and since the average vCJD spike was 10.9 RLU the sample was considered detectable with a S/CO of 3.8. The S/CO for samples with high PrP^{C} contaminant levels ranged from 0.8 to 1.4, suggesting borderline detection, while for low background samples S/CO was significantly higher at 2.2-8.3. Treatment of beads with Trypsin 50 μg/mL for 1 h at 37 C reduced contaminant load to background levels while preserving PrP^{SC} signal. As a result, the S/CO signal increased several fold to 2.3-5. We also observed that the background readings were decreased for samples that were considered low by an average of 1 RLU, resulting in an increased S/CO ratio. Thus treatment with trypsin reduced the background of un-spiked plasma from 21.3±31.6 to 1.3±0.6 RLU (Ave±SD).

This set of experiments demonstrated that trypsin can effectively digests PrP^{C} molecules that are contaminating the magnetic beads without affecting the levels of PrP^{Sc} that is bound to the PSR attached to the beads. This treatment reduces PrP^{C} contaminant of all samples to the same low background levels of 1-3 RLU resulting increase in detectability confidence.

TABLE 2

| | No Trypsin | | | | | With Trypsin | | | | |
|---|---|---|---|---|---|---|---|---|---|---|
| | Plasma + | | | | | Plasma + | | | | |
| | vCJD | | Plasma | | | vCJD | | Plasma | | |
| | Signal | | Bkgd | | | Signal | | Bkgd | | |
| Sample | (RLU) | SD | (RLU) | SD | S/CO | (RLU) | SD | (RLU) | SD | S/CO |
| N91835 | 10.9 | 0.3 | 2.1 | 0.3 | 3.8 | 6.7 | 1.0 | 0.6 | 0.0 | 9.4 |
| N91856 | 36.1 | 1.9 | 19.7 | 2.6 | 1.3 | 7.1 | 0.7 | 1.3 | 0.0 | 5.0 |
| N91858 | 120.6 | 12.4 | 114.3 | 2.3 | 1.0 | 8.1 | 0.9 | 2.7 | 0.3 | 2.3 |
| N91859 | 40.5 | 2.2 | 31.3 | 6.2 | 0.8 | 5.9 | 1.0 | 1.4 | 0.1 | 3.9 |
| N91860 | 43.8 | 3.6 | 36.9 | 1.0 | 1.1 | 7.8 | 0.2 | 1.3 | 0.1 | 4.7 |
| N91861 | 49.0 | 8.2 | 27.5 | 1.5 | 1.5 | 5.9 | 0.6 | 1.5 | 0.1 | 3.6 |
| N91863 | 45.7 | 4.7 | 41.9 | 2.8 | 0.9 | 9.9 | 0.4 | 2.3 | 0.3 | 3.2 |
| IPLA-2 | 28.7 | 3.3 | 15.0 | 1.7 | 1.4 | 6.5 | 0.4 | 1.4 | 0.1 | 3.9 |
| Pool 7 | 8.9 | 0.8 | 1.1 | 0.0 | 7.1 | 8.0 | 0.4 | 1.2 | 0.4 | 3.4 |
| Pool 8 | 10.0 | 0.7 | 0.8 | 0.1 | 8.3 | 8.9 | 0.5 | 0.8 | 0.1 | 8.6 |
| Pool 9 | 9.9 | 1.0 | 1.5 | 0.4 | 3.8 | 8.8 | 1.1 | 1.0 | 0.2 | 6.1 |
| Pool 10 | 13.3 | 1.7 | 2.0 | 0.5 | 3.9 | 9.6 | 0.6 | 0.9 | 0.1 | 7.0 |
| Pool 11-1 | 12.3 | 0.3 | 2.9 | 0.9 | 2.2 | 9.8 | 0.8 | 1.1 | 0.3 | 5.0 |
| Pool 11-2 | 9.9 | 0.9 | 1.7 | 0.4 | 3.3 | 7.9 | 0.5 | 0.9 | 0.2 | 5.4 |

### Example 3

### Protease Treatment of Human Brain Homogenates Containing PrP^{C} and PrP^{SC}

To determine if trypsin digestion would significantly reduce the signal from the PrP^{SC}, we studied the effect of trypsin on different CJD samples. Brain homogenates from three sporadic CJD patients (labeled; red, green, and yellow), two vCJD patients (white and blue) and normal controls were obtained from the National Institute of Biological Standard and Controls in the United Kingdom (NIBSC). The six brain homogenates were added into 200 μL of normal human plasmas (NHP pool 11, a plasma previously shown to have a low background level of PrP^{C}) at final concentration of 10 mL/mL and magnetic beads coated with a peptoid reagent as described in WO2007/030804 were added. After mixing for 1 hour at 37° C., the beads were washed and incubated with trypsin (50 μg/mL) at 37° C. for 1 hr. Trypsin digestion was stopped by adding 1 mM PMSF at room temperature for 10 min. Beads were washed again, subsequently denatured and detected by sandwich ELISA using 3F4 as capture and POM2 for detection as described in Examples 1 and 2.

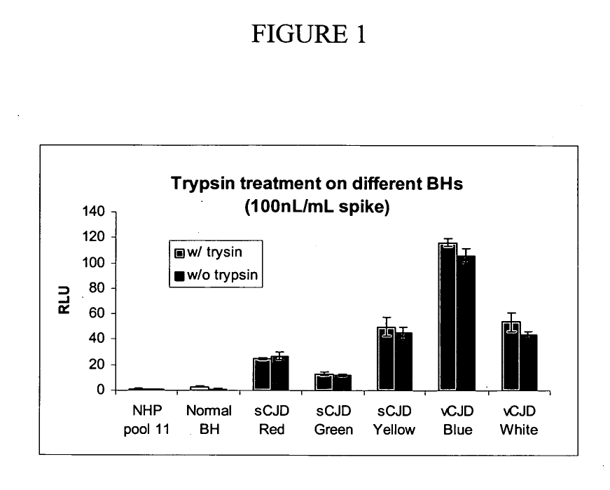

As shown in FIG. 1, PrP^{SC} was detected in all CJD homogenates and no differences in detection were observed after trypsin treatment, indicating that human PrP^{Sc} is generally resistant to trypsin digestion.

### Example 4

### Protease Treatment of Sheep Plasma Samples Containing PrP^{C} and PrP^{Sc}

The effect of protease treatment digestion on detection of PrP^{SC} in sheep PrP (shown in FIGS. 3 and 4, SEQ ID NO:5) was tested.

Plasma samples were treated with increasing concentrations of S-V8, trypsin or Proteinase K (PK). The samples were: 1) plasma from normal (that is, non-scrapie) sheep with low level of PrP^{C} (INR#1, 5 RLU), 2) normal sheep plasma with high level of PrP^{C} (224 L, 30 RLU) and 3) plasma spiked with brain homogenates from scrapie sheep (BH, 45 RLU). Samples were treated as described above in Example 2. Briefly, magnetic beads coated with a pathogenic prion-specific peptoid (PSR1) were added to the different samples (INR #1, 224 L or BH). After mixing for 1 hour at 37° C. beads were washed and incubated with one of three different proteases (trypsin, SV-8, or PK) at 37° C. for 1 hr. Protease digestion was stopped by adding PMSF to 1 mM to all of the samples at room temperature for 10 min. Beads were washed again, and subsequently denatured and detected by sandwich ELISA using POM19 as capture and POM2 for detection. POM19 recognizes an epitope in the sheep prion sequence at the carboxy terminal.

As shown in Table 3, treatment of INR#1 with the three different proteases did not reduce readings compared to untreated samples. However, treatment of 224 L with trypsin reduced readings from 30 RLU to a background level of 4.7 RLU, while digestion of plasma spiked with sheep brain PrP^{Sc} with trypsin 100 μg/mL did not reduce signal. PK digestion was not as efficient at 0.1 μg/mL concentration in reducing background due to PrP^{C} and also reduced the scrapie signal by 50%.

TABLE 3

| | | |
|---|---|---|
| INR#1 | 224L | BH |

| | Ave | SD | Ave | SD | Ave | SD |
|---|---|---|---|---|---|---|
| SV8 | | | | | | |
| 0 μg/mL | 5.1 | 0.1 | 30.8 | 1.5 | 45.1 | 2.0 |
| 100 μg/mL | 5.2 | 0.6 | 21.7 | 1.4 | 41.9 | 4.3 |
| 500 μg/mL | 4.7 | 0.4 | 14.0 | 1.0 | 37.7 | 9.0 |
| Trypsin | | | | | | |
| 0 μg/mL | 5.1 | 0.1 | 30.8 | 1.5 | 45.1 | 2.0 |
| 100 μg/mL | 3.9 | 0.2 | 4.7 | 0.2 | 41.6 | 9.7 |
| 500 μg/mL | 3.7 | 0.4 | 4.0 | 0.3 | 15.2 | 7.3 |
| PK | | | | | | |
| 0 μg/mL | 5.1 | 0.1 | 30.8 | 1.5 | 45.1 | 2.0 |
| 0.1 μg/assay | 4.2 | 0.0 | 10.2 | 6.9 | 21.0 | 3.7 |

### Example 5

### PK treatment to remove PrP^{C}

Recent reports have suggested that gentle treatment with proteinase K will efficiently eliminate PrP^{C} while preserving all or some of the octarepeat epitopes of PrP^{Sc} and therefore providing enhanced detection (See, U.S. Pat. No. 7,097,997 B1). In order to compare the effectiveness of PK treatment with use of the site-specific proteases, we carried out the following experiments.

PSR coated magnetic beads were incubated with different preparations of plasma containing high levels of PrP^{C}. 10% vCJD brain homogenate was spiked into low background plasma samples at final concentration of 40 nL/mL. Magnetic beads coated with peptoid (PSR1) were added to each sample. After mixing for 1 hour at 37° C., beads were washed and incubated with PK (0, 1, and 2 μg/mL) at 37° C. for 1 hr. Digestion was stopped by adding 2 mM PMSF at RT for 10 min. Beads were washed again, and subsequently denatured as described in Example 2 and detected by sandwich ELISA using 3F4 as capture antibody and POM2 antibody conjugated to Alkaline Phosphates (AP) for detection.

Levels of PrP^{C} background in the high PrP^{C} plasmas ranged from 4 to 35 RLU (rows 6-26), while the background of low PrP^{C} plasma was only 1.2 RLU (row 1). Spiking of the low PrP^{C} plasma with 40 nL/mL of vCJD 10% brain homogenate resulted in an increase of RLU from 1.2 to 9 RLU due to the presence of PrP^{Sc} in the vCJD brain homogenate (compare rows 1 and 2). In an attempt to reduce the background signal from the high PrP^{C} plasmas to that seen in the low PrP^{C} plasma, beads were treated with low concentrations of PK (1 and 2 μg/mL) for 1 h at 37° C.

To determine analytical sensitivity the ratio of signal to background was calculated (S/Co). To achieve statistical confidence of 99.7% the cutoff was defined as the average of the normal plasma plus three standard deviations (row 1), and ratios greater than 1.00 were considered positive. The cutoff was calculated to be 3.6 (1.2+3×0.8), and since the average vCJD spike was 9.3 RLU the sample was considered detectable with a S/CO of 2.6. The S/CO for samples with high contaminate levels ranged from 1 to 10, suggesting false positive detection. Treatment of beads with PK 1 μg/mL for 1 h at 37° C. did not affect detection of vCJD but also did not reduce the high background levels. Treatment with PK 2 μg/mL did not reduce background signal levels for the majority of the samples (rows 8, 11, 14, 17, 23 and 26) and S/CO values remained equal or above 1. In addition, even at this low concentration of PK, detection of the true positive signal (vCJD samples, rows 2-4) was reduced from 9 to 6 RLU and S/CO from 2.8 to 1.8, resulting loss of 35% in detection.

TABLE 4

| | Sample | PK (μg/mL) | RLU | SD | S/Co |
|---|---|---|---|---|---|
| 1 | Normal Plasma Background | 0 | 1.2 | 0.8 | |
| 2 | vCJD 40 nl/mL | 0 | 9.3 | 0.6 | 2.6 |
| 3 | | 1 | 10.1 | 0.2 | 2.8 |
| 4 | | 2 | 6.6 | 3.0 | 1.8 |
| 5 | High Background Normal Plasma | | | | |
| 6 | N91856 | 0 | 6.6 | 0.6 | 1.8 |
| 7 | | 1 | 6.9 | 0.5 | 1.9 |
| 8 | | 2 | 7.0 | 1.1 | 1.9 |
| 9 | N91858 | 0 | 35.0 | 3.5 | 9.7 |
| 10 | | 1 | 36.3 | 2.1 | 10.1 |
| 11 | | 2 | 36.8 | 4.5 | 10.2 |
| 12 | N91859 | 0 | 15.9 | 4.6 | 4.4 |
| 13 | | 1 | 9.3 | 0.7 | 2.6 |
| 14 | | 2 | 11.3 | 4.0 | 3.2 |
| 15 | N91860 | 0 | 10.8 | 0.8 | 3.0 |
| 16 | | 1 | 10.9 | 1.7 | 3.0 |
| 17 | | 2 | 10.6 | 0.8 | 3.0 |
| 18 | N91861 | 0 | 14.3 | 2.0 | 4.0 |
| 19 | | 1 | 14.9 | 0.6 | 4.1 |
| 20 | | 2 | 3.1 | 3.5 | 0.9 |
| 21 | N91863 | 0 | 10.0 | 1.6 | 2.8 |
| 22 | | 1 | 10.5 | 1.2 | 2.9 |
| 23 | | 2 | 7.5 | 5.0 | 2.1 |
| 24 | IPLA-2 | 0 | 3.5 | 0.8 | 1.0 |
| 25 | | 1 | 3.5 | 0.3 | 1.0 |
| 26 | | 2 | 3.5 | 2.0 | 1.0 |

In the next set of experiments we increased the concentration of PK to 4 and 8 μg/mL (Table 5). Increase of PK concentration to 4 μg/mL was enough to reduce the level of signal from the high PrP^{C} plasmas to background levels of 1.7-3 RLU. These experiments were done as described above but the PK concentration was increased to 4 μg/ml or 8 μg/ml.

TABLE 5

| | Normal Plasma | PK | | |
|---|---|---|---|---|
| | samples | (μg/mL) | RLU | SD |
| 1 | Normal Plasma | 0 | 3.1 | 0.6 |
| | Background | | | |
| 2 | N91835 | 0 | 3.2 | 0.1 |
| 3 | | 4 | 1.7 | 0.2 |
| 4 | | 8 | 1.8 | 0.3 |
| 5 | N91856 | 0 | 25.5 | 2.4 |
| 6 | | 4 | 4.3 | 0.3 |
| 7 | | 8 | 4.1 | 0.4 |
| 8 | N91858 | 0 | 68.4 | 6.8 |
| 9 | | 4 | 3.7 | 0.2 |
| 10 | | 8 | 2.9 | 0.2 |
| 11 | N91859 | 0 | 30.0 | 11.7 |
| 12 | | 4 | 5.8 | 2.4 |
| 13 | | 8 | 3.5 | 0.6 |
| 14 | N91860 | 0 | 16.1 | 2.0 |
| 15 | | 4 | 3.3 | 0.4 |
| 16 | | 8 | 2.9 | 0.5 |
| 17 | N91861 | 0 | 36.2 | 2.0 |
| 18 | | 4 | 3.0 | 0.2 |
| 19 | | 8 | 3.0 | 0.1 |
| 20 | N91863 | 0 | 33.4 | 3.8 |
| 21 | | 4 | 3.7 | 0.2 |
| 22 | | 8 | 2.6 | 0.1 |
| 23 | IPLA-2 | 0 | 11.2 | 2.6 |
| 24 | | 4 | 3.2 | 0.3 |
| 25 | | 8 | 3.5 | 0.8 |

Establishing that treatment of PSR-beads with PK at concentration of 4 μg/mL is effective in reducing levels of PrP^{C} contaminants we tested this treatment on vCJD PrP^{SC} attached to PSR-beads (Table 6). In this experiment increasing amounts of 10% vCJD brain homogenate (BH) were spiked into plasma and mixed with PSR coated-beads. Beads were treated with or without PK, proteins were denatured with NaOH as described in Example 2, 3 and PrP was detected with the sandwich ELISA as in Examples 2 and 3. As expected, increasing the concentration of vCJD BH resulted in higher detection signals (rows 1-5 at 0 μg/mL of PK). Treatment of PSR-beads with 4 μg/mL of PK resulted a 2-4 times drop in RLU and similar fold drop in the signal over background (S/CO) indicating a significant drop in sensitivity in detecting PrP^{SC}.

TABLE 6

| | | PK (μg/mL) | | | | | | |
|---|---|---|---|---|---|---|---|---|
| | vCJD spike | 0 | | | 4 | | | Fold Drop |
| | (nl/mL) | RLU | SD | S/CO | RLU | SD | S/CO | in S/CO |
| 1 | 0 | 1.4 | 0.1 | | 0.9 | 0.1 | | |
| 2 | 5 | 3.9 | 0.6 | 2.6 | 1.2 | 0.0 | 1.0 | 2.6 |
| 3 | 10 | 7.0 | 1.3 | 4.7 | 2.2 | 0.7 | 1.8 | 2.6 |
| 4 | 20 | 10.5 | 1.2 | 7.0 | 3.1 | 0.6 | 2.6 | 2.7 |
| 5 | 40 | 16.0 | 1.7 | 10.7 | 3.3 | 0.4 | 2.7 | 3.9 |

In the next set of experiments we studied the effect of PK on detection of vCJD PrP^{SC} using two different detection antibodies, POM2 (octarepeat region epitope) and POM17 (epitope is within the protease resistant fragment 90-231) (Polymenidou et al. Lancet, Vol 4, 804-814, 2005). Table 7 shows the results of this experiment. As seen in the experiments above, treatment with 4 μg/mL of PK resulted in an about 4-fold drop in sensitivity and a further increase in PK concentration resulted in a further drop in sensitivity when POM2 was used for detection. At similar PK concentrations, the drop in detection with POM17 was smaller since the 90-231 region is relatively resistant to PK. These results are consistent with what is known and expected about the protease resistant properties of PrP^{SC} residues 90-231 are resistant to mild protease digestion and therefore can be detected with antibodies that recognize epitopes within this region, while the N-terminus 23-90 is sensitive to PK and antibodies that bind the octarepeat region will lose their binding sites. This experiment also shows that treatment with increasing concentrations of PK results degradation of residues 90-231 as well, as RLU levels dropped from around 16 to 10 RLU even when POM17 was used for detection.

### Example 6

### Effect of Trypsin Concentration on Detection of Sheep PrP^{SC}

An ELISA plate (Microlite 2+) was coated with 150 μL of POM19 (3.3 μg/mL in 0.1M NaH₂PO₄.H₂O pH 6) overnight at 4° C. and blocked with 0.02% casein in TBST at 37° C. for 1 hour.

Normal sheep plasma was spiked with or without 250 mL/mL of 10% scrapie brain homogenates. In each well of a Greiner plate, 70 μL of plasma samples and 30 μL of 3.3×TBSTT (TBS, 1% Tween 20, and 1% Triton X-100) were incubated with 3 μL of PSR-beads (30 mg PSR/mL) at 37° C. for 2 hours with 750 rpm shaking. The beads were washed 4 times with TBST (TBS and 0.05% tween 20). Then 100 μL TBST with different concentrations of trypsin was added to each well. The plate was incubated at 37° C. for 30 minutes with 750 rpm shaking. PMSF (1 mM final) was added and incubated at room temperature for 10 min with 750 rpm shaking. The beads were washed again as above and then 75 μL of 0.1N NaOH at room temperature was added to each well for 10 minutes with 750 rpm shaking. The reactions were neutralized by 30 μL of 0.3N NaH₂PO₄ at room temperature for 5 minutes with 750 rpm shaking. To each well of the POM19-coated ELISA plate, 150 μL sample eluted from the beads was loaded and incubated at 37° C. for 1 hour with 300 rpm shaking.

POM2-AP (0.01 μg/mL in 0.001× casein-TBST) was used as detection antibody and incubated at 37° C. for 1 hr. Substrate (Lumi-Phos Plus and enhancer) was incubated at 37° C. for 30 min and detected by Luminoskan Ascent. The results are shown in Table 8. This example demonstrated that Trypsin concentrations as high as 100 μg/ml does not reduce the detection signal of PrP^{Sc}.

TABLE 7

| | | PK | POM2-AP | | | POM17-AP | | |
|---|---|---|---|---|---|---|---|---|
| | Sample | (μg/mL) | RLU | SD | S/CO | RLU | SD | S/CO |
| 1 | Normal Plasma | 0 | 1.5 | 0.5 | | 2.9 | 0.3 | |
| | Background | | | | | | | |
| 2 | vCJD 40 nl/mL | 0 | 13.0 | 1.7 | 4.3 | 15.9 | 0.4 | 4.2 |
| 3 | | 1 | 12.6 | 0.7 | 4.2 | 15.6 | 0.5 | 4.1 |
| 4 | | 2 | 11.7 | 0.4 | 3.9 | 13.9 | 1.6 | 3.7 |
| 5 | | 4 | 3.3 | 0.2 | 1.1 | 12.7 | 1.4 | 3.3 |
| 6 | | 8 | 1.8 | 0 | 0.6 | 10.2 | 0.9 | 2.7 |
| 7 | | 10 | 2.4 | 0.4 | 0.8 | 10.6 | 1 | 2.8 |

TABLE 8

| | Normal Sheep | | | Scrapie Brain | | |
|---|---|---|---|---|---|---|
| Trypsin | Plasma | | | Spiked Plasma | | |
| (μg/mL) | Ave | SD | % CV | Ave | SD | % CD |
| 500.0 | 3.70 | 0.39 | 10.48 | 15.24 | 7.25 | 47.59 |
| 100.0 | 3.88 | 0.15 | 3.94 | 41.56 | 9.70 | 23.34 |
| 0.0 | 5.14 | 0.11 | 2.12 | 45.10 | 1.95 | 4.32 |
| 100.0 | 5.02 | 0.24 | 4.72 | 51.84 | 4.51 | 8.70 |
| 33.33 | 4.35 | 0.24 | 5.46 | 43.40 | 17.23 | 39.70 |
| 11.11 | 4.89 | 0.45 | 9.18 | 60.05 | 7.70 | 12.82 |
| 3.70 | 4.07 | 0.02 | 0.54 | 43.85 | 15.15 | 34.55 |
| 1.23 | 4.57 | 0.46 | 10.09 | 50.23 | 23.49 | 46.76 |
| 0.41 | 4.58 | 0.31 | 6.79 | 50.75 | 20.00 | 39.41 |
| 0.00 | 6.50 | 0.58 | 8.95 | 56.71 | 25.63 | 45.19 |

Although preferred embodiments have been described in some detail, it is understood that obvious variations can be made without departing from the spirit and the scope of the disclosure as defined herein.

1. A method for detecting the presence of a pathogenic prion in a sample suspected of containing pathogenic and non-pathogenic prions, comprising
(a) contacting the sample with a pathogenic prion-specific reagent under conditions that allow binding of said reagent to said pathogenic prion, if present, to form a first complex;
(b) contacting said first complex with a site-specific protease under conditions in which the non-pathogenic prions are substantially digested by the protease;
(c) adding a protease inhibitor to prevent further cleavage by the site-specific protease;
(d) separating the first complex from any unbound sample and from cleaved non-pathogenic prions;
(e) dissociating said pathogenic prion from said first complex thereby providing dissociated pathogenic prion;
(f) contacting said dissociated pathogenic prion with a first anti-prion antibody under conditions that allow binding of said first anti-prion antibody to said pathogenic prion to form a second complex; and
(g) detecting formation of said second complex by contacting said second complex with a second anti-prion antibody, optionally labeled;
wherein said first anti-prion antibody recognizes a first epitope in said prion protein and said second anti-prion antibody recognizes a second epitope in said prion protein, wherein said first and second epitopes are not the same and are separated by at least one cleavage site for the site-specific protease, and wherein said at least one cleavage site for said site-specific protease is located within said proteinase K resistant core region of said prion protein.

2. The method of claim 1, wherein the site-specific protease comprises trypsin or SV8.

3. The method of claim 1 or 2, wherein the pathogenic-prion specific reagent is bound to a solid support.

4. The method of claim 3, wherein the solid support is a magnetic bead.

5. The method of claim 1, wherein the first anti-prion antibody is bound to a solid support.

6. The method of claim 5, wherein the first anti-prion antibody is bound to a microtiter plate.

7. The method of claim 1, wherein said dissociating step is carried out by exposing said first complex to high pH or low pH.

8. The method of claim 7, further comprising a step of neutralizing said high pH or said low pH following said dissociating step.

9. The method of claim 1, wherein said dissociated pathogenic prion is also denatured.

10. The method of claim 1, wherein either said first antibody or said second antibody recognizes an epitope in the octarepeat region of the prion protein.

11. The method of claim 10, wherein said antibody that recognizes an epitope in the octarepeat region is selected from the group consisting of POM2 and SAF-32.

12. The method of claim 1, wherein one of said first and second antibodies recognizes an epitope in the octarepeat region of said prion protein and the other antibody recognizes an epitope in the proteinase resistant core region of the prion protein.

13. The method of claim 12, wherein the antibody that recognizes an epitope in the proteinase K resistant core region is selected from the group consisting of 3F4, POM 17 and POM19.

14. The method of claim 1 wherein said protease inhibitor is phenylmethylsulfonyl fluoride.

15. In a method for detecting a pathogenic prion protein in a sample that also contains non-pathogenic prion proteins, the improvement comprises treating the sample with a site-specific protease under conditions in which the non-pathogenic prion proteins are substantially digested.


SEQUENCE LISTING (11 sequences)

SEQ ID NO: 1 - protein, 253 aa, Artificial Sequence
  Human prion protein
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 2 - protein, 244 aa, Artificial Sequence
  Monkey prion protein
MANLGCWMLVLFVATWSDLGLCKKRPKPGGWNTGGSRYPGGGSPGGNRYPPQGGGGWGQPHGGGWGQPHGGGWGQPHGGGWGQGGGTHNQWHKPSKPKTSMKHMAGAAAAGAVVGGLGGYMLGSAMSRPLIHFGNDYEDRYYRENMYRYPNQVYYRPVDQYSNQNNFVHDCVNITIKQHTVTTTTKGENFTETDVKMMERVVRQMCITQYEKESQAYYQRGSSMVLFSSPPVILLISFLFLIVG

SEQ ID NO: 3 - protein, 254 aa, Artificial Sequence
  Syrian hamster prion protein
MANLSYWLLALFVAMWTDVGLCKKRPKPGGWNTGGSRYPGQGSPGGNRYPPQGGGTWGQPHGGGWGQPHGGGWGQPHGGGWGQPHGGGWGQGGGTHNQWNKPSKPKTNMKHMAGAAAAGAVVGGLGGYMLGSAMSRPMMHFGNDWEDRYYRENMNRYPNQVYYRPVDQYNNQNNFVHDCVNITIKQHTVTTTTKGENFTETDIKIMERVVEQMCTTQYQKESQAYYDGRRSSAVLFSSPPVILLISFLIFLMVG

SEQ ID NO: 4 - protein, 264 aa, Artificial Sequence
  Bovine prion protein
MVKSHIGSWILVLFVAMWSDVGLCKKRPKPGGGWNTGGSRYPGQGXPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGXDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISFLIFLIVG

SEQ ID NO: 5 - protein, 256 aa, Artificial Sequence
  Sheep prion protein
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDRYSNQNNFVHDCVNITVKQHTVTTTTKGENFTETDIKIMERVVEQMCITQYQRESQAYYQRGASVILFSSPPVILLISFLIFLIVG

SEQ ID NO: 6 - protein, 254 aa, Artificial Sequence
  Mouse prion protein
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 7 - protein, 256 aa, Artificial Sequence
  Elk prion protein
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

SEQ ID NO: 8 - protein, 256 aa, Artificial Sequence
  Fallow deer prion protein
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMNRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

SEQ ID NO: 9 - protein, 256 aa, Artificial Sequence
  Mule deer prion protein
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGASVILFSSPPVILLISFLIFLIVG

SEQ ID NO: 10 - protein, 256 aa, Artificial Sequence; identical to SEQ ID NO: 9
  White-tailed deer prion protein

SEQ ID NO: 11 - protein, 9 aa, Artificial Sequence
  Octarepeat sequence
GQPHGGXGW